(19) United States
(12) Patent Application Publication
(10) Pub. No.: US 2019/0298290 A1
(43) Pub. Date: Oct. 3, 2019

(54) IMAGING SUPPORT APPARATUS AND RADIOGRAPHIC IMAGING SYSTEM

(71) Applicant: KONICA MINOLTA, INC., Tokyo (JP)

(72) Inventor: Sho NOJI, Tokyo (JP)

(21) Appl. No.: 16/361,632

(22) Filed: Mar. 22, 2019

(30) Foreign Application Priority Data
Mar. 29, 2018 (JP) 2018-063447

(51) Int. Cl. A61B 6/00

(52) U.S. Cl. CPC A61B 6/488; A61B 6/541

(57) ABSTRACT

An imaging support apparatus supports imaging by an imaging apparatus which radiographically images a dynamic state in a target site with a cyclic nature to obtain a dynamic image. The imaging support apparatus includes the following. Alive body information obtainer obtains live body information of an examined subject from a point before starting actual imaging with the imaging apparatus. A hardware processor evaluates stability for a cycle of the dynamic state in the target site based on the live body information obtained by the live body information obtainer.

### CROSS REFERENCE TO RELATED APPLICATIONS

The present application claims priority under 35 U.S.C. § 119 to Japanese Patent Application No. 2018-063447, filed on Mar. 29, 2018 is incorporated herein by reference in its entirety.

### BACKGROUND

### Technological Field

The present invention relates to an imaging support apparatus and a radiation imaging system.

### Description of the Related Art

Regarding imaging and diagnosis of a radiographic still image of a subject using conventional film/screen or a stimulable phosphor plate, there is an attempt to image a dynamic image of a subject using a semiconductor image sensor such as a flat panel detector (FPD) and applying the dynamic image to diagnosis. Specifically, speedy responsiveness in reading and deleting the image data of the semiconductor image sensor is used to successively irradiate pulsed radiation from a radiation source with the timing of reading and deleting of the semiconductor image sensor so that imaging is performed several times in one second. With this, the dynamic state of the subject is imaged.

Various techniques to support dynamic imaging are proposed. For example, Japanese Patent No. 3639826 describes a radiographic imaging system which outputs a guide to breathe such as “inhale” and “exhale” when the dynamic image of the chest portion is imaged, and the output of radiation starts when it is confirmed that the patient is breathing in a breathing cycle according to the breathing guide.

However, according to the technique described in Japanese Patent No. 3639826, the dynamic imaging is performed with the patient breathing at a cycle to match with the predetermined guide, and this is different from the patient's stable breathing cycle. Therefore, it is not possible to obtain the dynamic image in which the patient is stably breathing as normal.

### SUMMARY

The purpose of the present invention is to enable dynamic imaging in a state in which a cycle of a dynamic state is stable when a dynamic state in a target site which has a cyclic nature is imaged.

To achieve at least one of the above-mentioned objects, according to an aspect of the present invention, an imaging support apparatus reflecting one aspect of the present invention supports imaging by an imaging apparatus which radiographically images a dynamic state in a target site with a cyclic nature to obtain a dynamic image, the imaging support apparatus including: a live body information obtainer which obtains live body information of an examined subject from a point before starting actual imaging with the imaging apparatus; and a hardware processor which evaluates stability for a cycle of the dynamic state in the target site based on the live body information obtained by the live body information obtainer.

According to another aspect of the present invention, a radiographic imaging system includes: an imaging apparatus which radiographically images a dynamic state in a target site with a cyclic nature to obtain a dynamic image; and an imaging support apparatus.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram showing an entire configuration of a radiographic imaging system according to an embodiment of the present invention.

FIG. 2 is a flowchart showing an imaging control process A performed by a controller of an imaging console shown in FIG. 1 according to a first embodiment.

FIG. 3A is a diagram describing frequency, cycle, amplitude, and angular velocity.

FIG. 3B is a diagram describing a shift in a phase.

FIG. 3C is a diagram describing a curvature radius.

FIG. 4 is a diagram showing a table of conditions for a reference cycle (standard numeric range for the following, frequency, cycle, amplitude, angular velocity, shift in phase, index showing smoothness) stored in a storage.

FIG. 5 is a diagram schematically showing a specification of a reference cycle by a user.

FIG. 6A is a diagram showing an example of a stability evaluation screen when a cycle of a target site is stable.

FIG. 6B is a diagram showing an example of a stability evaluation screen when a cycle of a target site is unstable.

FIG. 7 is a diagram showing an example of displaying at the same time a waveform of cycle information obtained at present and a repeated waveform of a reference cycle on a stability evaluation screen.

FIG. 8 is a diagram showing an example of displaying a dynamic image and cycle information on different displays.

FIG. 9A is a diagram showing an example providing a display for displaying a dynamic cycle in a position where an examined subject is able to confirm the display by site easily when imaging is performed.

FIG. 9B is a diagram showing an example of display showing a display for displaying a dynamic cycle.

FIG. 10 is a diagram showing an example displaying a breathing guide for the examined subject on a display for displaying a dynamic cycle.

FIG. 11 is a flowchart showing an imaging control process B performed by a controller of an imaging console shown in FIG. 1 according to a second embodiment.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

### [Configuration of Radiographic Imaging System 100]

First, a configuration of a first embodiment of the present invention is described.

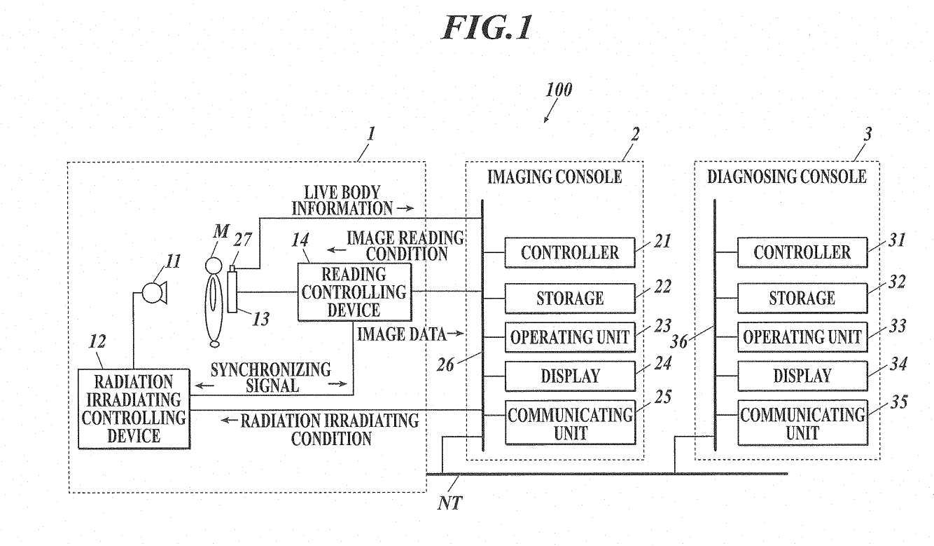

FIG. 1 is a diagram showing an entire configuration of a radiographic imaging system 100 according to the present embodiment.

As shown in FIG. 1, the radiographic imaging system 100 includes an imaging apparatus 1 connected to an imaging console 2 by a communication cable, and the imaging console 2 connected to a diagnosing console 3 through a communication network NT such as a LAN (Local Area Network). Each apparatus included in the radiographic imaging system 100 conforms to DICOM (Digital Image and Communications in Medicine) standards, and the communication among apparatuses is performed according to DICOM.

### [Configuration of Imaging Apparatus 1]

The imaging apparatus 1 is an imaging unit which images a dynamic state in a target site having a cyclic nature (cycle) such as a change in shape with the expansion and contraction of the lungs in the breathing movement and beating of the heart. In dynamic imaging, pulsed radiation such as X-rays are irradiated repeatedly in a predetermined time interval (pulsed irradiation) to a subject (target site in examined subject M) or the radiation is irradiated continuously at a low dosage rate (continuous irradiation) to the subject, and a plurality of images showing the dynamic state of the subject is obtained. A string of images obtained by dynamic imaging is called a dynamic image. Each of the plurality of images composing the dynamic image is called a frame image. The embodiments described below show an example of dynamic imaging by pulsed irradiation imaging the chest portion viewed from the front.

A radiation source 11 is positioned in a position facing a radiation detector 13 with a target site of an examined subject M in between, and radiation (X-ray) is irradiated to the target site according to control by the radiation irradiating controlling device 12.

A radiation irradiating controlling device 12 is connected to the imaging console 2, and the radiation source 11 is controlled based on radiation irradiating conditions input from the imaging console 2 to perform radiographic imaging. The radiation irradiating conditions input from the imaging console 2 include, for example, pulse rate, pulse width, pulse interval, number of imaged frames for each occasion of imaging, value of X-ray tube current, value of X-ray tube voltage, added filter type, etc. The pulse rate is the number of times the radiation is irradiated each second and matches with the later-described frame rate. The pulse width is the amount of time that radiation is irradiated for one irradiation of radiation. The pulse interval is the amount of time from the start of one irradiation of radiation to the start of the next irradiation of radiation, and this matches with the later described frame interval.

The radiation detector 13 includes a semiconductor image sensor such as a FPD (Flat Panel Detector), etc. The FPD includes a glass substrate, etc., for example. A plurality of detecting elements (pixels) are arranged in a matrix in a predetermined position on a substrate. The detecting elements detect radiation which is irradiated from the radiation source 11 and which passes at least the subject according to the strength of the radiation, convert the detected radiation to electric signals and accumulate the signals. The pixels include a switching unit such as a TFT (Thin Film Transistor). The type of the FPD may be an indirect conversion type which converts the X-ray to the electric signal through a scintillator by a photoelectric conversion element or a direct conversion type which directly converts the X-ray to the electric signal.

The radiation detector 13 is provided to face the radiation source 11 with the target site of the examined subject M in between.

A reading controlling device 14 is connected to an imaging console 2. The reading controlling device 14 controls the switching unit of each pixel in the radiation detector 13 based on the image reading conditions input from the imaging console 2 and switches the reading of the electric signals accumulated in each pixel. The electric signals accumulated in the radiation detector 13 are read to obtain the image data. This image data is the frame image. The reading controlling device 14 outputs the obtained frame image to the imaging console 2. The imaging reading conditions include, for example, frame rate, frame interval, pixel size, image size (matrix size), etc. The frame rate is the number of frame images obtained for one second, and matches with the pulse rate. The frame interval is the amount of time from the start of one operation to obtain the frame image to the start of the operation to obtain the next frame image, and matches with the pulse interval.

Here, the radiation irradiating controlling device 12 and the reading controlling device 14 are connected to each other. The devices communicate synchronizing signals in order to synchronize the radiation irradiating operation and the reading of the image.

### [Configuration of Imaging Console 2]

The imaging console 2 is an imaging support apparatus which supports imaging by the imaging apparatus 1. The radiation irradiating conditions and the image reading conditions are output to the imaging apparatus 1. With this, the radiation imaging and the reading operation of the radiographic image by the imaging apparatus 1 are controlled. The dynamic image obtained by the imaging apparatus 1 is displayed so that the operator performing the imaging such as an imaging technician is able to confirm positioning or confirm whether the image is suitable for diagnosis. The imaging console 2 obtains live body information of the examined subject M from before imaging to obtain the dynamic image for diagnosis (called actual imaging) is started, evaluates the stability in the cycle of the dynamic state in the target site, and displays the evaluation result.

As shown in FIG. 1, the imaging console 2 includes a controller 21, a storage 22, an operating unit 23, a display 24, and a communicating unit 25, and the above units are connected by a bus 26.

The controller 21 includes a CPU (Central Processing Unit), a RAM (Random Access Memory), etc. The CPU of the controller 21 reads the system program and various processing programs stored in the storage 22 according to the operation of the operating unit 23, and deploys the programs in the RAM. The CPU of the controller 21 executes various processes such as the later described imaging control process A according to the deployed program. The CPU of the controller 21 centrally controls the operation of each unit in the imaging console 2, and the radiation irradiating operation and the reading operation of the imaging apparatus 1.

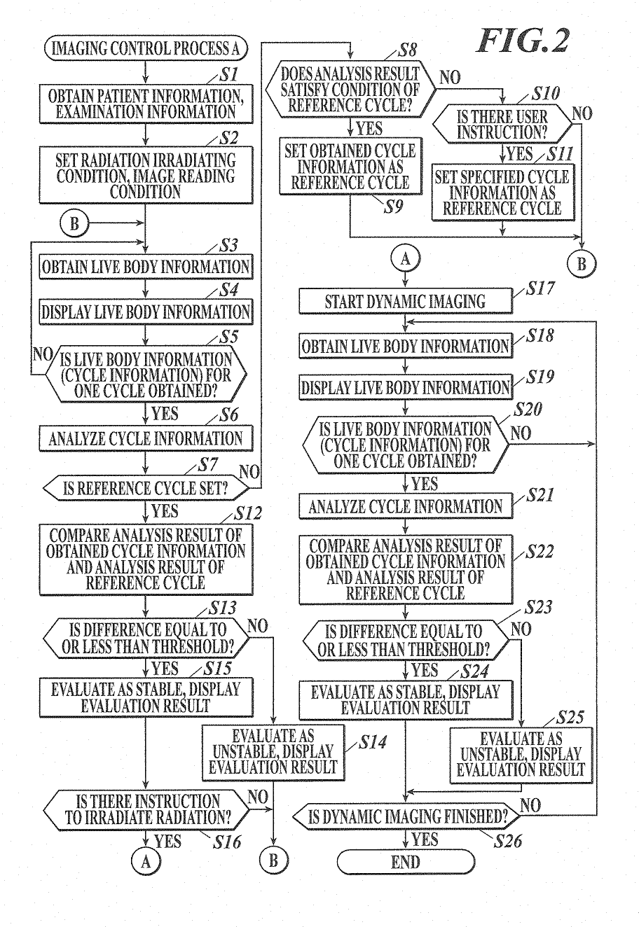

The storage 22 includes a nonvolatile semiconductor memory and a hard disk. The storage 22 stores various programs executed by the controller 21, parameters necessary to execute the process with the program or data showing the results of the process. For example, the storage 22 stores the program to execute the imaging control process A shown in FIG. 2. The storage 22 stores the radiation irradiating conditions and the image reading conditions in the case when the chest portion is imaged. Various programs are stored in a form of a readable program code, and the controller 21 performs the operation according to the program code.

The operating unit 23 includes a keyboard provided with cursor keys, numeral input keys, and various function keys and a pointing device such as a mouse. The operating unit 23 outputs to the controller 21 the instruction signal input by key operation on the keyboard and mouse operation. The operating unit 23 may include a touch panel on a display screen of the display 24, and in this case, the instruction signal input through the touch panel is output to the controller 21.

The display 24 includes a monitor such as a LCD (Liquid Crystal Display) or a CRT (Cathode Ray Tube), etc. and displays the instruction input on the operating unit 23 and data according to the instruction of the display signal input from the controller 21.

The communicating unit 25 includes a LAN adapter, a modem, or a TA (Terminal Adapter), and controls the transmitting and receiving of data between the devices connected to the communication network NT.

A live body information obtainer 27 obtains live body information of the examined subject M and outputs the information to the controller 21. The live body information obtained according to the present embodiment is the live body information showing the change in the dynamic state in the target site of the examined subject M. For example, the live body information may be the live body information which changes with the dynamic state in the target site. For example, when the target site is the lung field, the lung field moves with the breathing, and therefore, as the live body information obtainer 27, a breathing sensor, a spirometer, a breathing measuring device such as a breathing monitor belt, a camera which images the movement of the stomach, a sound collecting device such as a microphone which collects the sound generated from the examined subject (for example, breathing sound) can be used as the devices to measure the breathing movement. When the target site is the heart, as the live body information obtainer 27, a device which measures the heart beat such as an electrocardiograph can be used. An electromyography device can be used to measure the muscles which move together with the breathing or the heart beat. Preferably, the live body information obtainer 27 can be attached to the position which does not overlap with the target site in dynamic image capturing. For example, when the electrocardiograph is used, preferably, the device is attached to the arm.

### [Configuration of Diagnosing Console 3]

The diagnosing console 3 is a radiographic image analyzing apparatus which obtains the dynamic image from the imaging console 2, and displays the obtained dynamic image and the analysis result of the dynamic image in order to support the diagnosis by the physician.

As shown in FIG. 1, the diagnosing console 3 includes a controller 31, a storage 32, an operating unit 33, a display 34, and a communicating unit 35, and the above units are connected by a bus 36.

The controller 31 includes a CPU, a RAM, etc. The CPU of the controller 31 reads the system program and various processing programs stored in the storage 32 according to the operation of the operating unit 33, and deploys the programs in the RAM. The CPU of the controller 31 executes various processes according to the deployed program. The CPU of the controller 31 centrally controls the operation of each unit in the diagnosing console 3.

The storage 32 includes a nonvolatile semiconductor memory and a hard disk. The storage 32 stores various programs such as processing programs executed by the controller 31, parameters necessary to execute the process with the program or data showing the results of the process. Various programs are stored in a form of a readable program code, and the controller 31 performs the operation according to the program code.

The storage 32 stores the imaged dynamic image corresponded with patient information (for example, patient ID, patient name, height, weight, age, sex, etc.), examination information (for example, examination ID, examination date, subject site (here, chest portion), and imaging direction (front, side), etc.).

The operating unit 33 includes a keyboard provided with cursor keys, numeral input keys, and various function keys and a pointing device such as a mouse. The operating unit 33 outputs to the controller 31 the instruction signal input by key operation on the keyboard and mouse operation performed by the user. The operating unit 33 may include a touch panel on a display screen of the display 34, and in this case, the instruction signal input through the touch panel is output to the controller 31.

The display 34 includes a monitor such as a LCD or a CRT, etc. and displays various displays according to the instruction of the display signal input from the controller 31.

The communicating unit 35 includes a LAN adapter, a modem, or a TA, and controls the transmitting and receiving of data between the devices connected to the communication network NT.

### [Operation of Radiographic Imaging System 100]

Next, the operation of the radiographic imaging system 100 according to the present embodiment is described.

FIG. 2 shows a flowchart of the imaging control process A performed in the controller 21 of the imaging console 2. The imaging control process is executed by the controller 21 in coordination with the program stored in the storage 22.

First, the imaging operator operates the operating unit 23 of the imaging console 2 and inputs the patient information and the examination information for the examined subject M (step S1). After input, the imaging operator positions the target site of the examined subject M, attaches the live body information obtainer 27 to the examined subject M and instructs the breathing state (for example, instruction to breathe in a rested state).

Next, the radiation irradiating condition is read from the storage 22 and set in the radiation irradiating controlling device 12, and the image reading condition is read from the storage 22 and set in the reading controlling device 14 (step S2).

Next, the live body information is obtained by the live body information obtainer 27 (step S3).

The obtained live body information is stored in the RAM with the time information.

The live body information obtainer is not limited to the live body information obtainer 27 as described above. For example, from the dynamic image of the chest portion obtained by imaging the dynamic state of the chest portion with the imaging apparatus 1, when the target site is the lung field, the controller 21 measures the change in the square area of the lung field and the movement of the diaphragm (movement of the reference point of the diaphragm), and when the target site is the heart, the controller 21 measures the movement of the cardiac wall (movement of the reference point of the cardiac wall) and the change in the lung blood vessel diameter. The above can be obtained as the live body information. Structures such as the diaphragm, the lung field and the heart are imaged with clear contrast in the dynamic image. Therefore, when the dynamic state is imaged to obtain the live body information before performing the actual imaging, the measurement can be performed even if the dose is lowered greatly compared to the actual imaging. With this, from the viewpoint of suppressing irradiation, preferably, the controller 21 controls the radiation irradiating controlling device 12 so that the radiological amount is lower compared to actual imaging when the dynamic imaging to obtain the live body information is performed before the actual imaging. When the live body information is obtained by dynamic imaging, the imaging apparatus 1 is operated from before the actual imaging, and therefore a large sound (motor sound, etc.) is already generated due to operating the imaging device 1 before the actual imaging. Therefore, it is possible to reduce the possibility of the examined subject M being startled by the sound generated from the imaging apparatus 1 when the actual imaging starts and the stability of the cycle in the dynamic state of the target site being lost.

Here, for example, the square area of the lung field can be obtained from the frame images of the dynamic image by extracting the lung field region and multiplying the pixel size to the number of pixels in the extracted lung field region. For example, a threshold is obtained by discriminant analysis from a histogram of the signal value of each pixel and the region with the signals higher than this threshold is extracted primarily as the lung field region candidate. Next, the edge detection is performed near the boundary of the primarily extracted lung field candidate, and the points in which the edge is maximum in the small region near the boundary is extracted along the boundary. With this, the boundary of the lung field region can be extracted.

The position of the reference point of the diaphragm can be obtained by extracting the edge portion on the lower side of the lung field region in each frame image as the diaphragm boundary portion, setting a reference point in a position in one x-coordinate of the diaphragm boundary, and obtaining the y-coordinate of the set reference point.

The position of the reference point of the cardiac wall can be obtained by extracting the outline of the heart in each frame image, dividing the height of the extracted outline of the heart into three equal parts, determining the intersecting point of the horizontal line at the height of ⅓ from the bottom and the outline of the left ventricle as the reference point, and obtaining the x-coordinate of the reference point. A well-known method can be used to extract the outline of the heart, for example, the heart outline determining method as described in Japanese Patent No. 2796381.

The pulmonary vascular diameter can be obtained by extracting the blood vessel region from the lung field region in each frame image and obtaining the width of the extracted blood vessel region. As the process to extract the blood vessel, for example, a method to extract linear structures using the Hessian matrix can be used (for example, see Qiang Li, “Selective enhancement filters for nodules, vessels, and airway walls in two-and three-dimensional CT scans”, MEDICAL PHYSICS. SEPTEMBER 2003). It is difficult to acknowledge and capture arterioles with a thickness no larger than 0.3 mm, and the arterioles do not reflect the movement of the lung field compared to thick blood vessels. Preferably, the arterioles are removed from the extracted pulmonary vascular, the measuring points are provided on a main artery, artery, main vein and/or vein.

Next, the obtained live body information is displayed on the display 24 (step S4). For example, the obtained live body information is plotted on the time axis and displayed on the display 24.

Next, it is determined whether the live body information for one cycle (cycle information) is obtained (step S5). Here, the live body information for one cycle is cycle information showing a change of the dynamic state in the target site over time in one cycle. For example, based on the change over time of the live body information (waveform) sequentially obtained from the live body information obtainer 27, the timing of a predetermined feature point is set as the basic point of one cycle (for example, when the target site is the lung field, the converting point from inhale to exhale, the intermediate point between inhale and exhale, the converting point from exhale to inhale, etc.). The next time the feature point is detected, it is determined that the live body information is obtained for one cycle (that is, cycle information).

When it is determined that the live body information for one cycle (cycle information) is not obtained (step S5; NO), the process returns to step S3.

When it is determined that the live body information for one cycle (cycle information) is obtained (step S5; YES), the cycle information is analyzed and the analysis result is obtained for one or more of any of the following, frequency, cycle, amplitude, angular velocity, shift in phase, and index showing smoothness (step S6).

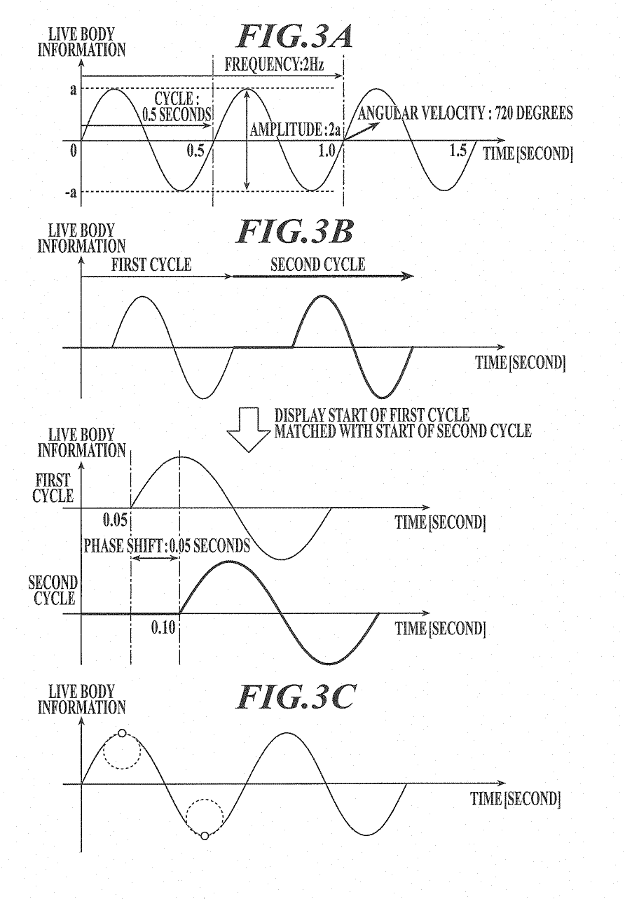

Here, as shown in FIG. 3A, the frequency is the number of times change (wave) is repeated in one second. The cycle is the amount of time from a state at one point to a point that the state returns to the state of the one point. The amplitude is the difference between the maximum value and the minimum value. The angular velocity is the angle advanced in one second. The shift in phase is the timewise shift of the waveform in each cycle as shown in FIG. 3B. The index showing smoothness is the value in which the curvature for each point on the waveform is calculated and which shows whether the curvature of each point is the same or the curvature changes at a certain percentage. For example, when the curvature of each point is the same or changes at a certain percentage, a predetermined index (for example, 1) showing that it is smooth is output. In other cases, a predetermined index (for example, 0) showing that it is not smooth is output. The curvature=1/curvature radius and the curvature radius is the radius of the circle closest to the curve (broken line circle in FIG. 3C).

Next, it is determined whether the reference cycle is already set (step S7). Here, the reference cycle is the cycle information in which the analysis result of the cycle information is within a preset standard value among the cycle information obtained from the examined subject M. When the reference cycle is already set, the cycle information of the reference cycle (reference waveform) and the analysis result are set in the predetermined region of the RAM in the controller 21.

When it is determined that the standard cycle is not obtained (step S7; NO), based on the analysis result of the cycle information, it is determined whether the obtained cycle information satisfies the condition of the reference cycle (step S8).

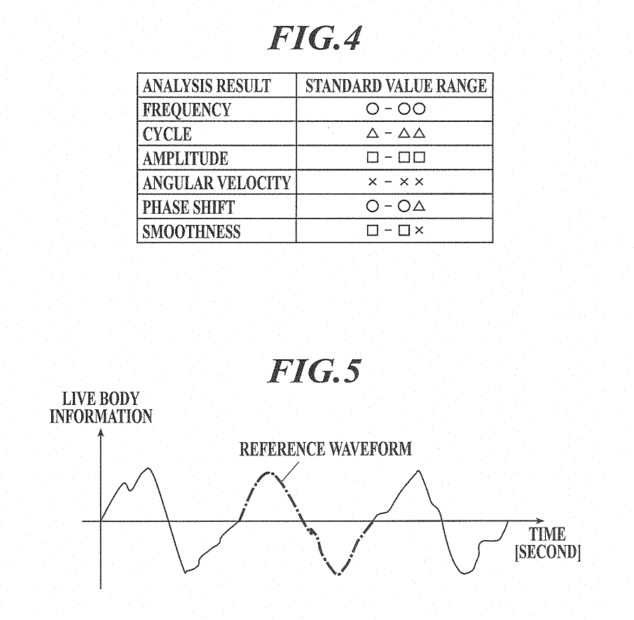

For example, the conditions of the reference cycle (the standard numeric range of the analysis result (frequency, cycle, amplitude, angular velocity, phase shift, index showing smoothness) of the cycle information when the dynamic state of the target site is stable) are stored in the storage 22 (see FIG. 4), and it is determined whether the obtained cycle information satisfy the conditions of the reference cycle based on the comparison between the analysis result of the obtained cycle information and the numeric range stored in the storage 22. At least one among the frequency, cycle, amplitude, angular velocity, phase shift and the index showing smoothness is to be used as the condition of the reference cycle, and all of the above do not have to be used.

When it is determined that the obtained cycle information satisfies the reference cycle conditions (step S8; YES), the obtained cycle information is automatically set as the reference cycle (step S9).

When it is determined that the obtained cycle information does not satisfy the reference cycle conditions (step S8; NO), it is determined whether the reference cycle is specified by operation on the operating unit 23 by the user (step S10).

Here, as shown in FIG. 5, when the stability in the cycle of the dynamic state in the target site is always disturbed by illness, all of the cycle information may not satisfy the reference cycle conditions. In the present embodiment, the user is able to specify on the operating unit 23 the waveform of the cycle which appears to be stable for the examined subject M from the cycle information (waveform) displayed on the display 24.

When it is determined that the reference cycle is specified by the user by operation on the operating unit 23 (step S10; YES), the specified cycle information is set as the reference cycle (step S11) and the process returns to step S3.

When it is determined that the reference cycle is not specified by the user by operation on the operating unit 23 (step S10; NO), the process returns to step S3.

In step S7, when it is determined that the reference cycle is already set (step S7; YES), the analysis result of the obtained cycle information is compared with the analysis result of the reference cycle (step S12).

As a result of the comparison, when it is determined that the difference between the analysis result of the obtained cycle information and the analysis result of the reference cycle exceeds the threshold set in advance (step S13; NO), it is evaluated that the cycle of the dynamic state in the target site is unstable, and a display showing that the cycle of the dynamic state in the target site is unstable is displayed on the display 24 (step S14). Then, the process returns to step S3. In step S14, preferably, a message notifying that it is not possible to perform the actual imaging is also displayed.

As a result of comparison, when it is determined that the difference between the analysis result of the obtained cycle and the analysis result of the reference cycle is equal to or less than the threshold value set in advance (step S13; YES), it is evaluated that the cycle of the dynamic state in the target site is stable, and the display showing that the cycle of the dynamic state in the target site is stable is displayed on the display 34 (step S15). Then, the process advances to step S16. In step S15, preferably, a message notifying that the actual imaging is possible (message urging start of the actual imaging) is also displayed.

As described above, according to the present embodiment, the stable cycle is set as the reference cycle from the cycle of the dynamic state in the target site in the examined subject M, and the cycle information sequentially obtained by the live body information obtainer 27 is compared with the reference cycle to evaluate the stability in the cycle of the dynamic state in the target site. Then, when the cycle of the dynamic state in the target site is not stable, the message notifying that the cycle is unstable and that the actual imaging cannot be performed is displayed. When the cycle is stable, the message notifying that the cycle is stable and urging the operator to start the actual imaging is displayed. Therefore, it is possible to prevent the actual imaging starting when the examined subject M is nervous and is in an unstable state different from the state in which the cycle of the dynamic state in the target site is stable for the examined subject M. With this, it is possible to avoid providing unnecessary irradiation on the examined subject M due to imaging the examined subject M again, and decrease in operation efficiency. It is possible to obtain the dynamic image in the state in which the cycle of the dynamic state in the target site is stable for the examined subject M.

Here, when the reference cycle is set by user operation in step S11, it is possible to evaluate stability according to the individual who is the examined subject M by enlarging the threshold used in step S13 and loosening the reference to determine whether the cycle of the dynamic state in the target site is stable compared to when the reference cycle is automatically set in step S9.

The information of the reference cycle (cycle information (waveform) and analysis result) set in the past can be stored in the storage 22 corresponded with the patient ID of the examined subject M, and the reference cycle set in the past for the same target site of the same examined subject and stored in the storage 22 can be set as the reference cycle as is.

In step S16, it is determined whether the radiation irradiating instruction is input on the operating unit 23 (step S16).

When it is determined that the radiation irradiating instruction is not input on the operating unit 23 (step S16; NO), the process returns to step S3, and steps S3 to S16 are repeatedly performed.

When it is determined that the radiation irradiating instruction is input on the operating unit 23 (step S16; YES), the imaging start instruction is output to the radiation irradiating controlling device 12 and the reading controlling device 14, and the dynamic imaging (actual imaging) is started (step S17). That is, the radiation is irradiated by the radiation source 11 at the pulsed interval set in the radiation irradiating controlling device 12, and the radiation detector 13 obtains the frame images. The frame images obtained by the actual imaging are sequentially input on the imaging console 2. The frame images are stored in the storage 22 corresponded with the number showing the order of imaging (frame number) and the frame images are displayed on the display 24.

Next, the live body information is obtained by the live body information obtainer 27 (step S18). The obtained live body information is stored in the RAM with the time information. The obtained live body information is not limited to those obtained from the live body information obtainer 27. As described in step S3, for example, the controller 21 can measure from the dynamic image obtained in the imaging apparatus 1 the change in the lung field square area, the movement of the diaphragm, the movement of the cardiac wall and the change in the pulmonary vascular diameter and obtain the above as the live body information.

Next, the obtained live body information is displayed on the display 24 (step S19). For example, the waveform plotting the obtained live body information on a time axis is displayed on the display 24. Here, preferably, the waveform obtained before the actual imaging is displayed so that this can be discriminated from the waveform obtained during imaging by displaying the timing of starting the actual imaging on the waveform or by displaying a description or using different colors for the waveform obtained before the actual imaging starts and the waveform obtained during the actual imaging. With this, the imaging operator can easily understand whether the cycle of the dynamic state in the target site is stable during the actual imaging.

Next, it is determined whether the live body information (cycle information) for one cycle is obtained (step S20). The method of determination in step S20 is similar to the method described in step S5, and therefore, the description is to be referred.

When it is determined that the live body information (cycle information) for one cycle is not obtained (step S20; NO), the process returns to step S18.

When it is determined that the live body information (cycle information) for one cycle is obtained (step S20; YES), the cycle information is analyzed, and the analysis result of at least one among the frequency, cycle, amplitude, angular velocity, phase shift and index showing smoothness is obtained (step S21). The analysis shown in step S21 is similar to the analysis described in step S6, and therefore, the description is to be referred.

Next, the analysis result of the obtained cycle information is compared with the analysis result of the reference cycle (step S22).

As a result of comparison, when it is determined that the difference between the analysis result of the obtained cycle and the analysis result of the reference cycle is equal to or lower than a predetermined threshold (step S23; YES), the cycle of the dynamic state in the target site is evaluated to be stable, and a display showing that the cycle of the dynamic state in the target site is stable is displayed on the display 24 (step S24). Then, the process advances to step S26. In step S24, a message notifying that the actual imaging can be continued can also be displayed.

As a result of comparison, when it is determined that the difference between the analysis result of the obtained cycle and the analysis result of the reference cycle exceeds the predetermined threshold (step S23; NO), it is determined that the cycle of the dynamic state in the target site is unstable, and a display showing that the cycle of the dynamic state in the target site is unstable is displayed on the display 34 (step S25). Then, the process advances to step S26. In step S25, preferably, a message notifying that the actual imaging cannot be continued is also displayed (message urging the operator to stop the imaging).

As described above, according to the present embodiment, the stable cycle from the cycle of the dynamic state in the target site in the examined subject M is set as the reference cycle, and the cycle information sequentially obtained by the live body information obtainer 27 during actual imaging is compared with the reference cycle to evaluate the stability of the cycle of the dynamic state in the target site. Then, when the cycle of the dynamic state in the target site is unstable, a display showing that the cycle is unstable or a message notifying that the actual imaging cannot be continued (message urging the operator to stop the imaging) is displayed. When the cycle of the dynamic state in the target site is stable, a display showing that the cycle is stable is displayed. Therefore, it is possible to prevent the actual imaging continuing as is when the examined subject M is nervous and is in an unstable state different from the state in which the cycle of the dynamic state in the target site is stable for the examined subject M. With this, it is possible to avoid providing unnecessary irradiation on the examined subject M due to imaging the examined subject M again, and decrease in operation efficiency. It is possible to obtain the dynamic image in the state in which the cycle of the dynamic state in the target site is stable for the examined subject M.

In step S26, it is determined whether the dynamic imaging is finished (step S26). For example, when the imaging of a predetermined number of frames is finished or to stop the imaging is instructed on the operating unit 23, it is determined that the dynamic imaging is finished.

When it is determined that the dynamic imaging is not finished (step S26; NO), the process returns to step S18, and the processes in step S18 to step S26 are repeatedly performed. When it is determined that the dynamic imaging is finished (step S26; YES), the imaging control process A ends.

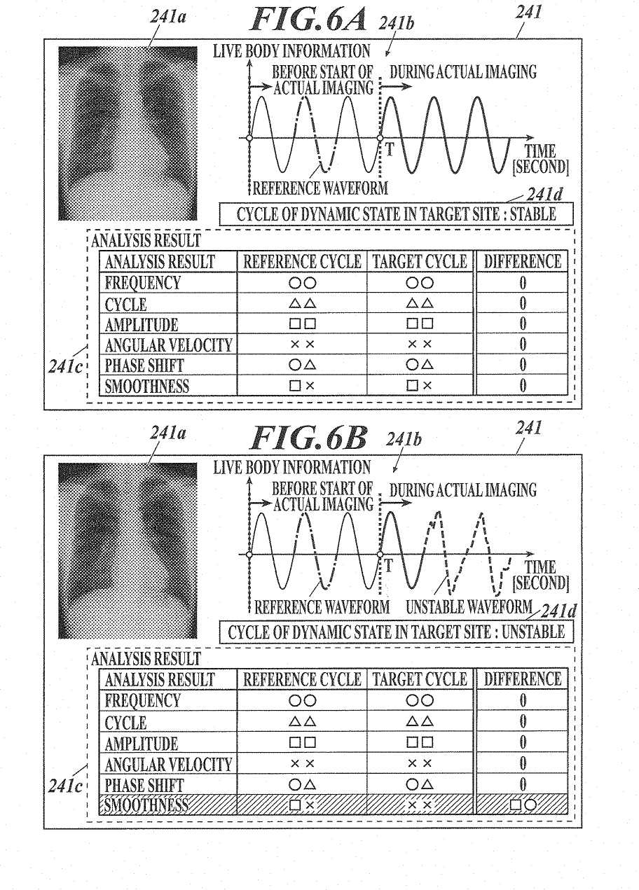

FIG. 6A is a diagram showing an example of a stability evaluation screen 241 displayed on the display 24 in step S24, and FIG. 6B is a diagram showing an example of a stability evaluation screen 241 displayed on the display 24 in step S25. As shown in FIG. 6A and FIG. 6B, the stability evaluation screen 241 displays the following, real-time dynamic image 241a obtained in the imaging apparatus 1, live body information (waveform) 241b obtained up to the present, analysis result 241c of the most recently obtained (real-time) cycle information, and evaluation result 241d of the most recently obtained (real-time) stability.

As described above, the cycle information 241b displays the waveform of the reference cycle so that this can be discriminated from the waveform of other cycles by displaying the reference cycle with a color different from the waveform of the other cycles or by displaying a description. Moreover, the cycle information 241b displays the waveform obtained before the actual imaging starts so that this can be discriminated from the waveform obtained during the actual imaging by displaying the timing T showing the start of the actual imaging on the waveform, by displaying the waveform obtained before the start of the actual imaging with a color different from the waveform obtained after the start of the actual imaging (during imaging) or by displaying a description. The analysis result 241c of the cycle information displays at least the analysis result of the reference cycle and the analysis result of the latest cycle information and the difference value of the above. The value in the analysis result 241c of the cycle information is updated each time the new cycle information is taken in.

When the unstable waveform is generated during the actual imaging, as shown in FIG. 6B, the stability evaluation result 241d displays that the waveform is unstable. Further, the cycle information 241b displays the unstable waveform with a color different from the stable waveform or displays a description so that the unstable waveform can be discriminated from the stable waveform. At the same time, preferably, the unstable waveform is notified to the imaging operator by flashing the screen of the display 24 or emitting a warning sound. Preferably, the color of the letters showing the item of the analysis result or the color of the lines of the frame forming the table determined to be unstable is displayed with the color changed in the analysis result 241c of the cycle information.

As described above, in the stability evaluation screen 241, the waveform of the reference cycle in the stable state obtained from the dynamic state in the target site in the examined subject himself and the waveform of the cycle information for the dynamic state in the target site obtained by the live body information obtainer 27 are displayed. Therefore, the imaging operator is able to intuitively understand the present state of the cycle of the dynamic state in the target site by comparison with the reference cycle. The waveform before the start of the actual imaging is displayed so that this can be discriminated from the waveform during the actual imaging. Therefore, the imaging operator is able to easily understand whether the cycle of the dynamic state in the target site during the actual imaging is stable. The analysis result of the reference cycle, the analysis result of the latest cycle, and the difference value are displayed in a numeric value. Therefore, the difference from the reference cycle can be confirmed with a numeric value.

When the unstable waveform is generated, other than displaying that the waveform is unstable, the waveform of the unstable cycle is displayed with a color different from the waveform of the stable cycle and the letters or the colors of the analysis result item determined to be unstable are changed. Therefore, it is possible to easily understand how different the obtained cycle is from the reference cycle and which items are unstable.

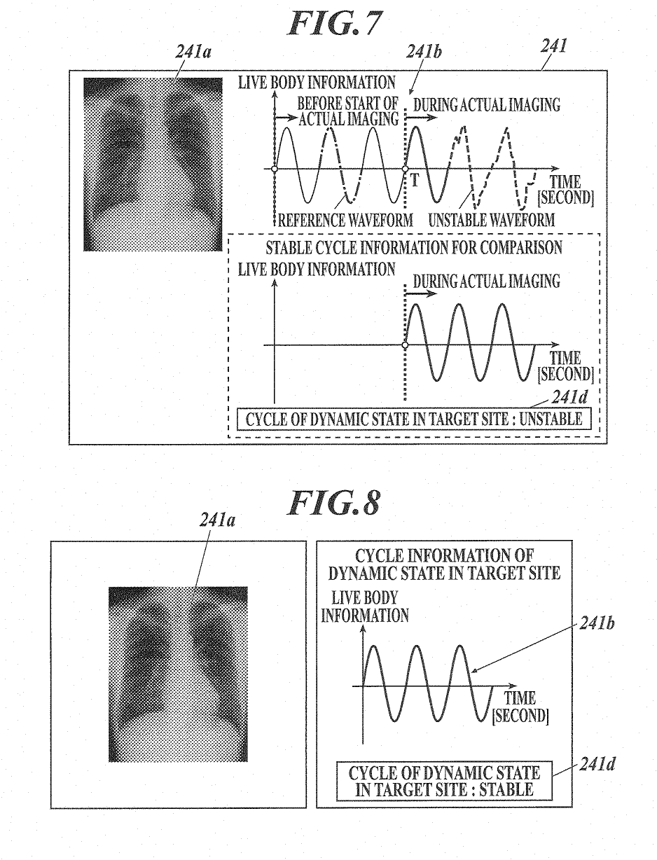

In order to show the difference between the reference cycle in a stable state and the presently obtained cycle in a form that can be easily understood, as shown in FIG. 7, at least during the dynamic imaging, the waveform of the reference cycle is repeated and displayed at the same time as the waveform of the presently obtained cycle on the stability evaluation screen 241. With this, the imaging operator is able to more easily understand the difference between the reference cycle and the presently obtained cycle.

As shown in FIG. 8, the dynamic image 241a and the cycle information 241b in the stability evaluation screen 241 can be displayed on separate displays. With this, for example, even if the screen size of the display 24 is small, it is possible to prevent decrease in the visibility of the cycle of the dynamic state in the target site.

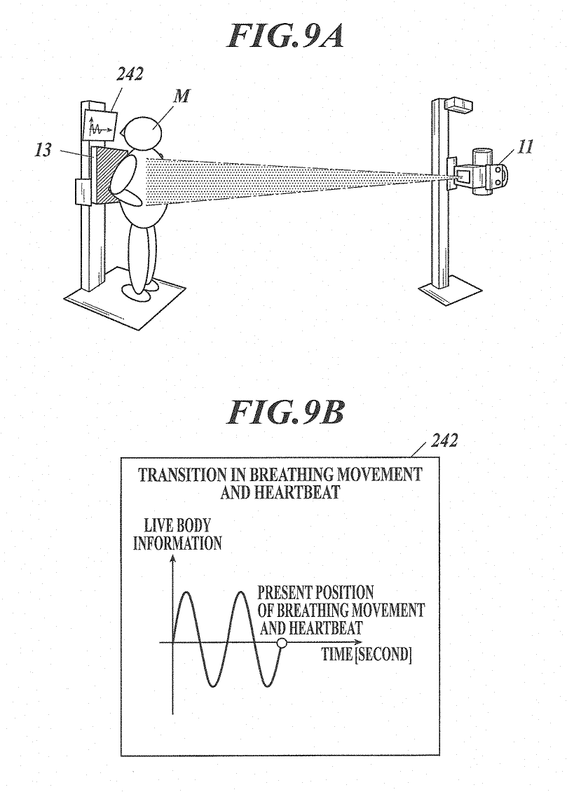

As shown in FIG. 9, a dynamic cycle display 242 can be provided in a position where the examined subject M can easily confirm by sight when the imaging is performed, on an imaging stage or behind the imaging stage, for example. The controller 21 may display in real time the waveform of the obtained live body information (cycle information of the dynamic state in the target site) on not only the display 24 but also the dynamic cycle display 242 (see FIG. 9B). With this, the examined subject M can also confirm whether the dynamic state in the target site is in a stable state.

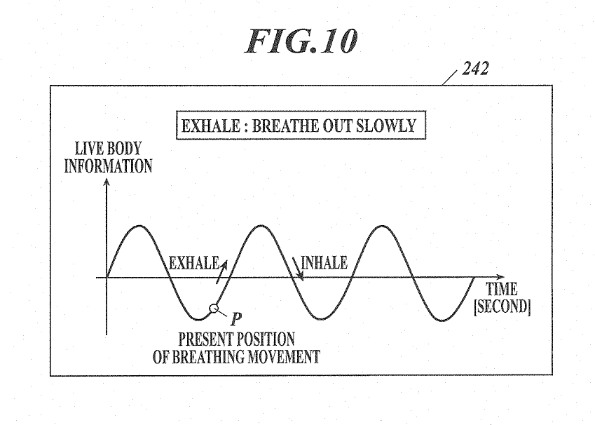

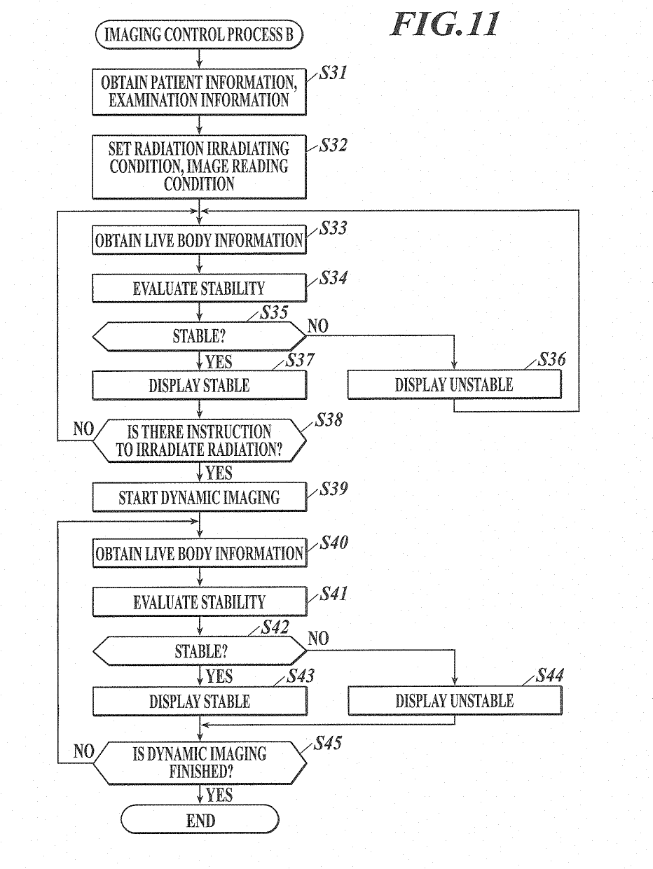

When the target site is mainly the lungs, the controller 21 may repeatedly display on the dynamic cycle display 242 the waveform of the reference cycle which is the cycle when the breathing of the examined subject M is stable so that the examined subject M is guided to breathe stably. For example, when the movement of the diaphragm is obtained as the live body information, as shown in FIG. 10, the present position P of the breathing cycle is displayed on the waveform displaying the waveform of the reference cycle repeatedly. Moreover, when the waveform is rising “Exhale: Breathe out slowly” is displayed and when the waveform is falling “Inhale: Breathe in slowly” is displayed on the dynamic cycle display 242 as the breathing guide. Moreover, the imaging console 2 may include a sound output unit so that the breathing can be guided by an automatic voice according to the present waveform position. Specifically, when the waveform is rising “Exhale: Breathe out slowly” and when the waveform is falling “Inhale: Breathe in slowly” are output by the automatic voice as the breathing guide. Since the reference cycle is obtained before the actual imaging, the predetermined voice can be set in advance to be output automatically according to the rise/fall of the waveform of the reference cycle, and the breathing guide according to the waveform position P is output by the automatic voice. The display and the sound of the breathing guide can be combined. Further, the color of the screen and/or the color of the waveform can be changed to match the exhale phase and the inhale phase. Alternatively, an animated movie showing breathing can be displayed.

As described above, by guiding the breathing based on the reference cycle obtained from the live body information of the examined subject M himself, it is possible to guide the examined subject M to his usual stable breathing cycle when an unstable breathing cycle can be seen due to the examined subject M being nervous. In conventional breathing guides, the guide uses the information of the breathing cycle which does not belong to the examined subject himself. Therefore, the examined subject M is guided to a cycle different from the stable breathing cycle of the examined subject M. Since the breathing guide is performed based on the reference cycle obtained from the breathing cycle of the examined subject M himself, it is possible to guide the examined subject M to the usual stable breathing cycle.

When the dynamic imaging is finished, information such as identification ID to identify the dynamic image, patient information, examination information, radiation irradiating condition, image reading condition, and number showing order of imaging (frame number) is added to each of the string of frame images obtained in the dynamic imaging (for example, written in the head region of the image data in a DICOM form) and the result is transmitted to the diagnostic console 3 through the communicating unit 25.

In the diagnosing console 3, when the string of frame images of the dynamic image is received from the imaging console 2, the received dynamic image is stored in the storage 32 corresponded with the patient information and the examination information. Then, the dynamic image is selected on the operating unit 33, and when the display is instructed, the controller 31 reads the selected dynamic image from the storage 32 and displays the image on the display 34. When the dynamic image is selected on the operating unit 33 and the analysis is instructed, the controller 31 reads the selected dynamic image from the storage 32 and performs the analyzing process. The analysis result is displayed on the display 34. As the analysis of the chest portion dynamic image, for example, Japanese Patent Application Laid-Open Publication No 2012-110451 describes ventilation analysis and blood flow analysis.

### Second Embodiment

Next, the second embodiment of the present invention is described.

According to the second embodiment, the storage 22 of the imaging console 2 stores the program to perform the imaging control process B shown in FIG. 11.

According to the second embodiment, the storage 2 of the imaging console 2 stores the program to perform the imaging control process shown in FIG. 11.

Moreover, the live body information obtainer 27 according to the second embodiment obtains live body information regarding at least one of the following, body temperature of the examined subject M, perspiration amount, body shake amount, breathing rate, heart rate, etc. The obtained live body information is output to the controller 21. The following can be used as the live body information obtainer 27 according to the second embodiment, for example, a thermometer, perspiration meter, vibration measuring sensor, breathing sensor, spirometer, respiratory meter such as a breathing monitor belt, a camera which images movement of the stomach, respiratory sound measurement, and electrocardiogram.

Other components of the second embodiment are similar to those described in the first embodiment, and therefore, the description is omitted. The operation of the second embodiment is described below.

FIG. 11 shows a flowchart describing the imaging control process B performed in the controller 21 of the imaging console 2 according to the second embodiment. The imaging control process B is performed by the controller 21 in coordination with the program stored in the storage 22.

First, the imaging operator operates the operating unit 23 of the imaging console 2, and input of the patient information of the examined subject M and the examination information is performed (step S31). After input, the imaging operator positions the examined subject M and instructs the breathing state (for example, instructs to breathe in the rested state).

Next, the radiation irradiating condition is read from the storage 22 and set in the radiation irradiating controlling device 12, and the image reading condition is read from the storage 22 and set in the reading controlling device 14 (step S32).

Next, the live body information obtainer 27 obtains the live body information (step S33).

For example, the live body information obtains at least one of the following, body temperature of the examined subject M, perspiration amount, body shake amount, breathing rate, heart rate, etc.

Next, the stability of the cycle of the dynamic state in the target site is evaluated based on the obtained live body information (step S34).

In step S34, the degree of nervousness of the examined subject M is determined using an AI (Artificial Intelligence) based on the live body information of at least one of the following body temperature of the examined subject M, perspiration amount, body shake amount, breathing rate, heart rate, etc. Since the breathing cycle and the heart beat cycle are not stable if the degree of nervousness is high (that is, the dynamic state of the lungs and the heart do not become stable), it is evaluated that when the degree of nervousness is a predetermined threshold or lower, the target site is evaluated to be stable, and when the degree of nervousness exceeds the predetermined threshold, the target site is evaluated to be unstable.

As the AI, for example, deep learning which is a field of machine learning can be used. For example, deep learning can learn the relation between one live body information set or a combination of a plurality of live body information sets and the degree of nervousness of the examined subject when the live body information is obtained. In step S34, the obtained live body information can be input in the deep learning to obtain the degree of nervousness of the examined subject M.

Next, it is determined whether the result of evaluating the stability of the cycle of the dynamic state in the target site is determined (step S35).

When it is determined that the evaluation result of the stability is unstable (step S35; NO), the display showing that the cycle of the dynamic state in the target site is unstable is displayed on the display 24 (step S36), and the process returns to step S33. In step S33, preferably, a message notifying that the actual imaging cannot be performed is also displayed.

As described above, when it is evaluated that the cycle of the dynamic state in the target site is unstable, this evaluation is displayed. Therefore, it is possible to prevent the actual imaging being performed in the state in which the cycle of the dynamic state in the target site is unstable. Further, it is possible to avoid providing unnecessary irradiation on the examined subject M due to imaging the examined subject M again, and decrease in operation efficiency.

When it is determined that the evaluation result of the stability is stable (step S35; YES), it is displayed that the cycle of the dynamic state in the target site is stable on the display 34 (step S37), and the process advances to step S38. In step S37, preferably, the message notifying that the actual imaging is possible is also displayed (message urging start of actual imaging).

As described above, when it is evaluated that the cycle of the dynamic state in the target site is stable, this evaluation is displayed and the start of the actual imaging is urged. Therefore, it is possible to obtain the dynamic image in the state that the cycle of the dynamic state in the target site is stable.

In step S38, it is determined whether the radiation irradiating instruction is input on the operating unit 23 (step S38).

When it is determined that the radiation irradiating instruction is input on the operating unit 23 (step S38; YES), the imaging start instruction is output to the radiation irradiating controlling device 12 and the reading controlling device 14, and the dynamic imaging (actual imaging) is started (step S39). That is, when the radiation is irradiated from the radiation source 11 at the pulse interval set in the radiation irradiating controlling device 12, the radiation detector 13 obtains the frame images. The frame images obtained by the actual imaging are sequentially input on the imaging console 2. The frame images are stored in the storage 22 corresponded with the number showing the order of imaging (frame number) and the frame images are displayed on the display 24.

Next, the live body information obtainer 27 obtains the live body information (step S40), and based on the obtained live body information, the stability of the cycle of the dynamic state in the target site is evaluated (step S41). The method to evaluate the stability in step S41 is similar to the method described in step S34, and the description is to be referred.

Next, it is determined whether the stability evaluation result is stable (step S42).

When it is determined that the stability evaluation result is stable (step S42; YES), the display showing that the cycle of the dynamic state in the target site is stable is displayed on the display 24 (step S43), and the process advances to step S45. In step S43, a message notifying that the actual imaging can be continued can also be displayed.

When it is determined that the stability evaluation result is not stable (step S42; NO), the display showing that the cycle of the dynamic state in the target site is unstable is displayed on the display 34 (step S44), and the process advances to step S45. In step S44, preferably, a message notifying that the actual imaging cannot be continued (message urging the operator to stop the imaging) is also displayed.

As described above, when the cycle of the dynamic state in the target site is unstable during the actual imaging, the display showing that the cycle is unstable is displayed and the operator is urged to stop imaging. Therefore, it is possible to prevent the imaging continuing in the state in which the cycle of the dynamic state in the target site is unstable. With this, it is possible to avoid unnecessary irradiation on the examined subject M, and decrease in operation efficiency. It is possible to obtain the dynamic image in the state in which the cycle of the dynamic state in the target site is stable.

In step S45, it is determined whether the dynamic imaging is finished (step S45). For example, when the imaging of a predetermined number of frames is finished or to stop the imaging is instructed on the operating unit 23, it is determined that the dynamic imaging is finished.

When it is determined that the dynamic imaging is not finished (step S45; NO), the process returns to step S40, and the processes in step S40 to step S45 are repeatedly performed. When it is determined that the dynamic imaging is finished (step S45; YES), the imaging control process B ends.

When the dynamic imaging is finished, information such as identification ID to identify the dynamic image, patient information, examination information, radiation irradiating condition, image reading condition, and number showing order of imaging (frame number) is added to each of the string of frame images obtained in the dynamic imaging (for example, written in the header region of the image data in a DICOM form) and the result is transmitted to the diagnostic console 3 through the communicating unit 25.

The operation of the diagnosing console 3 is similar to the operation described in the first embodiment, and therefore the description is referred

According to the imaging control process B, the stability of the dynamic state in the target site is evaluated based on live body information using the AI. Alternatively, the live body information at a predetermined point can be set as the reference live body information, the set reference live body information can be compared with the live body information obtained by the live body information obtainer 27, and the stability of the dynamic state in the target site can be evaluated based on the compared result. For example, the predetermined point can be the point after a predetermined amount of time passes from the start of obtaining the live body information. Alternatively, the table showing the conditions of the reference live body information (standard numeric range of the live body information when the dynamic state in the target site is stable) is stored in the storage 22, and the predetermined point can be the point when live body information obtained from the live body information obtainer 27 satisfies the condition of the reference live body information.

As described above, according to the controller 21 of the imaging console 2, the live body information obtainer 27 obtains the live body information of the examined subject from a point before the actual imaging by the imaging apparatus 1 starts and the stability is evaluated for the cycle of the dynamic state in the target site with a cyclic nature based on the obtained live body information.

Therefore, when the dynamic state of the target site with the cyclic nature is imaged, the imaging operator is able to confirm the result of evaluation showing the stability of the cycle of the dynamic state in the target site and is able to perform the dynamic imaging in a state in which the cycle of the dynamic state is stable.

The description according to the above-described embodiments are merely examples of the present invention, and the present invention is not limited to the above.

For example, according to the first embodiment, the stability of the cycle of the dynamic state in the target site is evaluated based on the result of analyzing the cycle information of the dynamic state in the target site. Alternatively, when the target site is the lung field, the type of breathing (breathing in rested state, deep breathing, or held breath) can be determined based on the analysis result of the cycle information for the square area of the lung field or the movement of the diaphragm, and the determined result can be displayed on the display 24. For example, when the amplitude of the cycle information (waveform) for the movement of the diaphragm is 0.2 cm or more and less than 1.3 cm, the controller 21 determines that it is breathing in a rested state, when it is 1.3 cm or more, it is determined that it is deep breathing, and when it is less than 0.2 cm, it is determined that the breath is being held. With this, the imaging operator can easily understand whether the examined subject is able to perform the desired breathing.

The cycle information obtained before the actual imaging starts and after the actual imaging starts can be obtained by a different live body obtainer 27 if the information is the same cycle information. For example, the breathing cycle information corresponding to the cycle information of the movement of the lungs can be obtained with the respiratory sensor before the actual imaging starts, and the cycle information of the movement of the diaphragm corresponding to the cycle information of the dynamic state of the lungs can be obtained from the dynamic image after the actual imaging starts.

When the stability is evaluated based on the result of analyzing the obtained cycle information of the dynamic state in the target site, the evaluation is performed based on the comparison between the analysis result of the cycle information of the reference cycle and the analysis result of the obtained cycle information. Alternatively, the AI can be used to evaluate the stability based on the obtained cycle information of the dynamic state in the target site.

According to the present embodiment, the target site is the dynamic state of the lung field and the heart, but the target site may be a joint or other portion.

For example, according to the above description, a hard disk or a nonvolatile semiconductor memory is used as the computer-readable medium storing the program regarding the present invention, but the present invention is not limited to the above. For example, as other computer-readable mediums, a portable recording medium such as a CD-ROM can be applied. A carrier wave may be applied as the medium providing the data of the program according to the present invention through communication lines.

The detailed configuration and the detailed operation of the devices included in the radiographic imaging system can be suitably changed without leaving the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An imaging support apparatus which supports imaging by an imaging apparatus which radiographically images a dynamic state in a target site with a cyclic nature to obtain a dynamic image, the imaging support apparatus comprising:
a live body information obtainer which obtains live body information of an examined subject from a point before starting actual imaging with the imaging apparatus; and
a hardware processor which evaluates stability for a cycle of the dynamic state in the target site based on the live body information obtained by the live body information obtainer.

2. The imaging support apparatus according to claim 1, wherein the live body information obtainer obtains at least one of the following live body information, body temperature of the examined subject, perspiration amount, body shake amount, breathing rate, and heart rate.

3. The imaging support apparatus according to claim 1, wherein,
the live body information obtainer obtains cycle information showing a change over time for each cycle of the dynamic state in the target site as the live body information,
the hardware processor analyzes the cycle information of the dynamic state in the target site obtained by the live body information obtainer; and
the hardware processor evaluates the stability for the cycle of the dynamic state in the target site based on a result of the above analysis.

4. The imaging support apparatus according to claim 1, wherein the live body information obtainer is a sensor which obtains live body information of the examined subject.

5. The imaging support apparatus according to claim 3, wherein the live body information obtainer is a hardware processor which controls the imaging apparatus to obtain a chest portion dynamic image at a radiological dose lower than a dose in the actual imaging in order to obtain the cycle information before actual imaging starts, and when the target site is the lungs, cycle information of a movement of a diaphragm or a change in a lung field square area is obtained as the live body information from the chest portion dynamic image imaged by the imaging apparatus and when the target site is a heart, the cycle information of a movement of a cardiac wall or a change in a pulmonary vascular diameter is obtained as the live body information from the chest portion dynamic image imaged by the imaging apparatus.

6. The imaging support apparatus according to claim 3, wherein the hardware processor analyzes the cycle information of the dynamic state in the target site and obtains for each cycle at least one of the following, a frequency, a cycle, an amplitude, an angular velocity, a phase shift, and an index showing smoothness of a waveform.

7. The imaging support apparatus according to claim 1, wherein the hardware processor evaluates the stability for the cycle of the dynamic state in the target site based on the live body information by using artificial intelligence.

8. The imaging support apparatus according to claim 1, wherein the hardware processor sets the live body information at a predetermined point from the live body information obtained by the live body information obtainer as reference live body information and evaluates the stability for the cycle of the dynamic state in the target site based on a comparison between the live body information obtained by the live body information obtainer and the reference live body information.

9. The imaging support apparatus according to claim 3, wherein the hardware processor sets a reference cycle from the cycle information obtained by the live body information obtainer and evaluates the stability for a cycle of the dynamic state in the target site based on a comparison between an analysis result of the cycle information obtained by the live body information obtainer and an analysis result of the cycle information of the reference cycle.

10. The imaging support apparatus according to claim 9, further comprising a storage which stores a range of a standard value in an analysis result of the cycle information,
wherein the hardware processor automatically sets the reference cycle based on a comparison between an analysis result of the cycle information obtained by the live body information obtainer and a range of the standard value in the analysis result of the cycle information stored in the storage.

11. The imaging support apparatus according to claim 9, wherein the hardware processor sets a cycle specified by user operation from a cycle included in the cycle information obtained by the live body information obtainer as the reference cycle.

12. The imaging support apparatus according to claim 9, wherein the hardware processor evaluates the stability for the cycle of the dynamic state in the target site before starting the actual imaging based on a comparison between an analysis result of the cycle information obtained before starting the actual imaging and an analysis result of the reference cycle, and evaluates the stability for the cycle of the dynamic state in the target site during the actual imaging based on a comparison between an analysis result of the cycle information obtained during the actual imaging and an analysis result of the reference cycle.

13. The imaging support apparatus according to claim 1, further comprising a display which displays an evaluation result showing stability for the cycle of the dynamic state in the target site.

14. The imaging support apparatus according to claim 13, wherein the display is provided in a position which can be confirmed by sight by an operator when the imaging by the imaging apparatus is performed.

15. The imaging support apparatus according to claim 13, wherein the display is provided in a position which can be confirmed by sight by an examined subject when the imaging by the imaging apparatus is performed.

16. The imaging support apparatus according to claim 15, wherein the display displays a waveform of the reference cycle in the evaluation showing the stability.

17. The imaging support apparatus according to claim 16, further comprising an output unit which outputs a breathing guide to the examined subject based on a waveform of the reference cycle displayed on the display.

18. The imaging support apparatus according to claim 13, wherein the display displays information showing whether the actual imaging can be started or continued based on the waveform of the cycle information of the dynamic state in the target site, an analysis result of the cycle information of the dynamic state in the target site and/or the evaluation result showing stability.

19. The imaging support apparatus according to claim 16, wherein the display simultaneously displays the waveform of the cycle information of the dynamic state in the target site obtained by the live body information obtainer during the actual imaging and the waveform based on the reference cycle.

20. A radiographic imaging system comprising:
an imaging apparatus which radiographically images a dynamic state in a target site with a cyclic nature to obtain a dynamic image; and
an imaging support apparatus according to claim 1.
